SEQUENCE LISTING (31 sequences)

SEQ ID NO: 1 - protein, 8 aa, Artificial Sequence
  Synthetic
DYKDDDDK

SEQ ID NO: 2 - DNA, 366 bp, Mus musculus
cagatccagttggtgcagtctggacctgagctgaagaaccctggagagacagtcaagatctcctgcaaggcttctgggtataccttcacaaactatggaatgaactgggtgaagcaggctccaggaaagggtttaaagtggatggggtggataaacacccacactggagagccaacatatgctgatgacttcaagggacggtttgccttctcttcggaaacctctgccagctctgcctatttgcagatcaacaacctcaaaaatgatgacatggctacatatttctgtgcaaaaggtacccacagagaagaaattccggcctggttcgcttactggggccaagggactctggtcactgtctctgca

SEQ ID NO: 3 - protein, 122 aa, Mus musculus
QIQLVQSGPELKNPGETVKISCKASGYTFTNYGMNWVKQAPGKGLKWMGWINTHTGEPTYADDFKGRFAFSSETSASSAYLQINNLKNDDMATYFCAKGTHREEIPAWFAYWGQGTLVTVSA

SEQ ID NO: 4 - DNA, 339 bp, Mus musculus
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccagagctcctgatctacaaagtttccaacctattttctggggtcccagacaggttcagtggcagtggatcagggacagatttcacactcaagatcagcagagtggaggctgaggatctgggagtttattactgctttcaaggttcacatgttcctcccacgttcggtgctgggaccaagctggagctgaaacgg

SEQ ID NO: 5 - protein, 113 aa, Mus musculus
DVLMTQTPLSLPVSLGDQASISCRSSQSIVHSNGNTYLEWYLQKPGQSPELLIYKVSNLFSGVPDRFSGSGSGTDFTLKISRVEAEDLGVYYCFQGSHVPPTFGAGTKLELKR

SEQ ID NO: 6 - DNA, 45 bp, Artificial Sequence
  Synthetic
ggtggcggtggttctggtggcggtggttctggtggcggtggttct

SEQ ID NO: 7 - protein, 15 aa, Artificial Sequence
  Synthetic
GGGGSGGGGSGGGGS

SEQ ID NO: 8 - DNA, 750 bp, Mus musculus
cagatccagttggtgcagtctggacctgagctgaagaaccctggagagacagtcaagatctcctgcaaggcttctgggtataccttcacaaactatggaatgaactgggtgaagcaggctccaggaaagggtttaaagtggatggggtggataaacacccacactggagagccaacatatgctgatgacttcaagggacggtttgccttctcttcggaaacctctgccagctctgcctatttgcagatcaacaacctcaaaaatgatgacatggctacatatttctgtgcaaaaggtacccacagagaagaaattccggcctggttcgcttactggggccaagggactctggtcactgtctctgcaggtggcggtggttctggtggcggtggttctggtggcggtggttctgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccagagctcctgatctacaaagtttccaacctattttctggggtcccagacaggttcagtggcagtggatcagggacagatttcacactcaagatcagcagagtggaggctgaggatctgggagtttattactgctttcaaggttcacatgttcctcccacgttcggtgctgggaccaagctggagctgaaacgg

SEQ ID NO: 9 - protein, 250 aa, Mus musculus
QIQLVQSGPELKNPGETVKISCKASGYTFTNYGMNWVKQAPGKGLKWMGWINTHTGEPTYADDFKGRFAFSSETSASSAYLQINNLKNDDMATYFCAKGTHREEIPAWFAYWGQGTLVTVSAGGGGSGGGGSGGGGSDVLMTQTPLSLPVSLGDQASISCRSSQSIVHSNGNTYLEWYLQKPGQSPELLIYKVSNLFSGVPDRFSGSGSGTDFTLKISRVEAEDLGVYYCFQGSHVPPTFGAGTKLELKR

SEQ ID NO: 10 - DNA, 63 bp, Homo sapiens
atggccttaccagtgaccgccttgctcctgccgctggccttgctgctccacgccgccaggccg

SEQ ID NO: 11 - protein, 21 aa, Homo sapiens
MALPVTALLLPLALLLHAARP

SEQ ID NO: 12 - DNA, 24 bp, Artificial Sequence
  Synthetic
gactacaaagacgatgacgacaag

SEQ ID NO: 13 - DNA, 141 bp, Homo sapiens
aagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgat

SEQ ID NO: 14 - protein, 47 aa, Homo sapiens
KPTTTPAPRPPTPAPTIASQPLSLRPEASRPAAGGAVHTRGLDFASD

SEQ ID NO: 15 - DNA, 204 bp, Homo sapiens
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 16 - protein, 68 aa, Homo sapiens
FWVLVVVGGVLACYSLLVTVAFIIFWVRSKRSRLLHSDYMNMTPRRPGPTRKHYQPYAPPRDFAAYRS

SEQ ID NO: 17 - DNA, 342 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 18 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - DNA, 1542 bp, Mus musculus
atggccttaccagtgaccgccttgctcctgccgctggccttgctgctccacgccgccaggccggctagccagatccagttggtgcagtctggacctgagctgaagaaccctggagagacagtcaagatctcctgcaaggcttctgggtataccttcacaaactatggaatgaactgggtgaagcaggctccaggaaagggtttaaagtggatggggtggataaacacccacactggagagccaacatatgctgatgacttcaagggacggtttgccttctcttcggaaacctctgccagctctgcctatttgcagatcaacaacctcaaaaatgatgacatggctacatatttctgtgcaaaaggtacccacagagaagaaattccggcctggttcgcttactggggccaagggactctggtcactgtctctgcaggtggcggtggttctggtggcggtggttctggtggcggtggttctgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccagagctcctgatctacaaagtttccaacctattttctggggtcccagacaggttcagtggcagtggatcagggacagatttcacactcaagatcagcagagtggaggctgaggatctgggagtttattactgctttcaaggttcacatgttcctcccacgttcggtgctgggaccaagctggagctgaaacgggactacaaagacgatgacgacaagctcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 20 - protein, 512 aa, Mus musculus
MALPVTALLLPLALLLHAARPASQIQLVQSGPELKNPGETVKISCKASGYTFTNYGMNWVKQAPGKGLKWMGWINTHTGEPTYADDFKGRFAFSSETSASSAYLQINNLKNDDMATYFCAKGTHREEIPAWFAYWGQGTLVTVSAGGGGSGGGGSGGGGSDVLMTQTPLSLPVSLGDQASISCRSSQSIVHSNGNTYLEWYLQKPGQSPELLIYKVSNLFSGVPDRFSGSGSGTDFTLKISRVEAEDLGVYYCFQGSHVPPTFGAGTKLELKRDYKDDDDKLEKPTTTPAPRPPTPAPTIASQPLSLRPEASRPAAGGAVHTRGLDFASDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPP

SEQ ID NO: 21 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 22 - protein, 42 aa, Homo sapiens
KRGRKKLLYIFKQPFMRPVQTTQEEDGCSCRFPEEEEGGCEL

SEQ ID NO: 23 - DNA, 1521 bp, Mus musculus
atggccttaccagtgaccgccttgctcctgccgctggccttgctgctccacgccgccaggccggctagccagatccagttggtgcagtctggacctgagctgaagaaccctggagagacagtcaagatctcctgcaaggcttctgggtataccttcacaaactatggaatgaactgggtgaagcaggctccaggaaagggtttaaagtggatggggtggataaacacccacactggagagccaacatatgctgatgacttcaagggacggtttgccttctcttcggaaacctctgccagctctgcctatttgcagatcaacaacctcaaaaatgatgacatggctacatatttctgtgcaaaaggtacccacagagaagaaattccggcctggttcgcttactggggccaagggactctggtcactgtctctgcaggtggcggtggttctggtggcggtggttctggtggcggtggttctgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccagagctcctgatctacaaagtttccaacctattttctggggtcccagacaggttcagtggcagtggatcagggacagatttcacactcaagatcagcagagtggaggctgaggatctgggagtttattactgctttcaaggttcacatgttcctcccacgttcggtgctgggaccaagctggagctgaaacggctcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 24 - protein, 506 aa, Mus musculus
MALPVTALLLPLALLLHAARPASQIQLVQSGPELKNPGETVKISCKASGYTFTNYGMNWVKQAPGKGLKWMGWINTHTGEPTYADDFKGRFAFSSETSASSAYLQINNLKNDDMATYFCAKGTHREEIPAWFAYWGQGTLVTVSAGGGGSGGGGSGGGGSDVLMTQTPLSLPVSLGDQASISCRSSQSIVHSNGNTYLEWYLQKPGQSPELLIYKVSNLFSGVPDRFSGSGSGTDFTLKISRVEAEDLGVYYCFQGSHVPPTFGAGTKLELKRLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - DNA, 360 bp, Homo sapiens
gaagtgcagctgctggaaagcggcggcggcctggtgcagccgggcggcagcctgcgcctgagctgcgcggcgagcggctttacctttagcagctatattatgatgtgggtgcgccaggcgccgggcaaaggcctggaatgggtgagcagcatttatccgagcggcggcattaccttttatgcggataccgtgaaaggccgctttaccattagccgcgataacagcaaaaacaccctgtatctgcagatgaacagcctgcgcgcggaagataccgcggtgtattattgcgcgcgcattaaactgggcaccgtgaccaccgtggattattggggccagggcaccctggtgaccgtgagcagc

SEQ ID NO: 26 - protein, 120 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 27 - DNA, 45 bp, Artificial Sequence
  Synthetic
ggcggcggcggcagcggcggcggcggcagcggcggcggcggcagc

SEQ ID NO: 28 - DNA, 330 bp, Homo sapiens
cagagcgcgctgacccagccggcgagcgtgagcggcagcccgggccagagcattaccattagctgcaccggcaccagcagcgatgtgggcggctataactatgtgagctggtatcagcagcatccgggcaaagcgccgaaactgatgatttatgatgtgagcaaccgcccgagcggcgtgagcaaccgctttagcggcagcaaaagcggcaacaccgcgagcctgaccattagcggcctgcaggcggaagatgaagcggattattattgcagcagctataccagcagcagcacccgcgtgtttggcaccggcaccaaagtgaccgtgctg

SEQ ID NO: 29 - protein, 112 aa, Homo sapiens
QSALTQPASVSGSPGQSITISCTGTSSDVGGYNYVSWYQQHPGKAPKLMETIYDVSNRPSGVSNRFSGSKSGNTASLTISGLQAEDEADYYCSSYTSSSTRVFGTGTKVTVL

SEQ ID NO: 30 - DNA, 735 bp, Homo sapiens
gaagtgcagctgctggaaagcggcggcggcctggtgcagccgggcggcagcctgcgcctgagctgcgcggcgagcggctttacctttagcagctatattatgatgtgggtgcgccaggcgccgggcaaaggcctggaatgggtgagcagcatttatccgagcggcggcattaccttttatgcggataccgtgaaaggccgctttaccattagccgcgataacagcaaaaacaccctgtatctgcagatgaacagcctgcgcgcggaagataccgcggtgtattattgcgcgcgcattaaactgggcaccgtgaccaccgtggattattggggccagggcaccctggtgaccgtgagcagcggcggcggcggcagcggcggcggcggcagcggcggcggcggcagccagagcgcgctgacccagccggcgagcgtgagcggcagcccgggccagagcattaccattagctgcaccggcaccagcagcgatgtgggcggctataactatgtgagctggtatcagcagcatccgggcaaagcgccgaaactgatgatttatgatgtgagcaaccgcccgagcggcgtgagcaaccgctttagcggcagcaaaagcggcaacaccgcgagcctgaccattagcggcctgcaggcggaagatgaagcggattattattgcagcagctataccagcagcagcacccgcgtgtttggcaccggcaccaaagtgaccgtgctg

SEQ ID NO: 31 - protein, 245 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

(19) United States
(12) Patent Application Publication
(10) Pub. No.: US 2023/0227557 A1
(43) Pub. Date: Jul. 20, 2023

(54) PD-L1-SPECIFIC ANTIBODY AND ANTI-PD-L1-CAR-T CELLS

(71) Applicants: ProMab Biotechnologies, Inc., Richmond, CA (US); Forevertek Biotechnology Co., Ltd, Changsha (CN)

(72) Inventors: Lijun Wu, Albany, CA (US); Vita Golubovskaya, Richmond, CA (US)

(21) Appl. No.: 18/002,340

(22) PCT Filed: Jun. 16, 2021
(86) PCT No.: PCT/US2021/037573

§ 371 Date: Dec. 19, 2022

Related U.S. Application Data
(60) Provisional application No. 63/044,115, filed on Jun. 25, 2020.

(51) Int. Cl. C07K 16/28; C07K 14/705; C07K 14/725

(52) U.S. Cl. CPC C07K 16/2827; C07K 14/70521; C07K 14/7051; C07K 14/70578; C07K 2317/21; C07K 2317/622; C07K 2319/33; C07K 2317/76; C07K 2319/03; C07K 2317/73

(57) ABSTRACT

The present invention is directed to a monoclonal anti-human PD-L1 antibody, or a single-chain variable fragment (scFv), comprising V_{H} having the amino acid of SEQ ID NO: 3 and V_{L} having the amino acid of SEQ ID NO: 5. The present invention is also directed to a chimeric antigen receptor fusion protein comprising from N-terminus to C-terminus: (i) a single-chain variable fragment (scFv) of the present invention, (ii) a transmembrane domain, (iii) at least one co-stimulatory domains, and (iv) an activating domain. The inventors have shown that the PD-L1 CAR-T cells of the present invention are more effective than Avelumab PD-L1 CAR-T cells in killing several cancer cell lines. PD-L1 CAR-T can be used alone or in combination with other agent in an immunotherapy.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence Listing.txt with a creation date of Jun. 15, 2021, and a size of 31.1 kilobytes. The Sequence Listing filed via EFS-Web is part of the specification and is hereby incorporated in its entirety by reference herein.

### FIELD OF THE INVENTION

The present invention relates to PD-L1 (programmed death ligand-1)-specific antibody and anti-PD-L1-CAR-T Cells, which are useful in the field of adoptive immunity gene therapy for tumors.

### BACKGROUND OF THE INVENTION

Immunotherapy is emerging as a highly promising approach for the treatment of cancer. T cells or T lymphocytes, the armed forces of our immune system, constantly look for foreign antigens and discriminate abnormal (cancer or infected cells) from normal cells. Genetically modifying T cells with CAR (Chimeric antigen receptor) constructs is the most common approach to design tumor-specific T cells. CAR-T cells targeting tumor-associated antigens (TAA) can be infused into patients (called adoptive cell transfer or ACT) representing an efficient immunotherapy approach [1, 2]. The advantage of CAR-T technology compared with chemotherapy or antibody is that reprogrammed engineered T cells can proliferate and persist in the patient (“a living drug”)[1, 3, 4].

CARs usually consist of a monoclonal antibody-derived single-chain variable fragment (scFv) at the N-terminal part, hinge, transmembrane domain and a number of intracellular co-activation domains: (i) CD28, (ii) CD137 (4-1BB), CD27 or other co-stimulatory domains, in tandem with an activation CD3-zeta domain. (FIG. 1) [1, 2]. The evolution of CARs went from first generation (with no co-stimulation domains) to second generation (with one co-stimulation domain) to third generation CAR (with several co-stimulation domains). Generating CARs with multiple costimulatory domains (the so-called 3^{rd} generation CAR) have led to increased cytolytic CAR-T cell activity, improved persistence of CAR-T cells leading to its augmented antitumor activity.

FIG. 1 shows the structures of CAR. The left panel shows the structure of first generation (no co-stimulation domains), the middle panel shows the second generation (one co-stimulation domain CD28 or 4-1BB), and the right panel shows the third generation of CAR (two or several co-stimulation domains). The Figure is from Golubovskaya, Wu, Cancers, 2016[5].

PD-L1, also known as cluster of differentiation 274, CD274 or B7 homolog 1, B7-H1, is a 40 kDa transmembrane protein that plays a significant role in suppressing an immune system during disease or other events. The binding of PD-L1 domain to PD-1 protein blocks proliferation and activity of CD8+ T cells, responsible for immune defense. The PD-L1/PD-1 interaction plays a dominant role in the suppression of T cell responses in vivo, especially in the tumor microenvironment.

Several types of cancer overexpress PD-L1. Anti-PD-L1 monoclonal antibodies (mAbs) and anti-PD-1 mAbs immunotherapies are tested in clinical trials [3]. PD-L1 is usually not expressed in normal tissues on the cell surface, but its expression is elevated in many tumor tissues. In addition, PD-L1 expression is significantly up-regulated by immune cells mainly through their production of IFN-gamma.

The U.S. Food and Drug Administration (FDA) granted accelerated approval to avelumab in 2017, for the treatment of adults and pediatric patients 12 years and older with metastatic Merkel cell carcinoma. Avelumab is a PD-L1-blocking human IgG1 lambda monoclonal antibody.

There exists a need for an anti-PD-L1 antibody having high specificity and activity.

### BRIEF DESCRIPTION OF THE DRAWINGS

FIG. 1. The structures of CAR

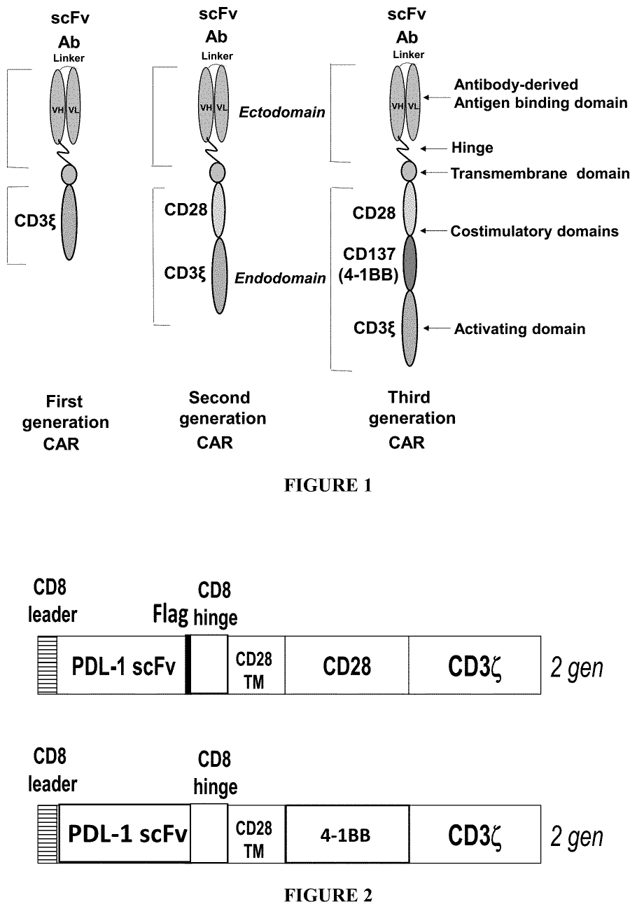

FIG. 2. The structures of PD-L1 CAR construct with either CD28 (top) or 4-1BB (bottom) as a co-stimulating domain. FLAG tag is optional.

FIG. 3. PD-L1 antibody detects PD-L1 antigen by ELISA and by Western blotting.

FIG. 4. FACS with PD-L1 antibody using different cancer cell lines. HepG2, SKOV-3 express high level of PD-L1.

FIG. 5A-5E. Promab PD-L1-CAR-T cells (PMC159, CD28; PMC804, 4-1BB) are highly cytotoxic against PD-L1-positive cancer cells. Effector to target cells ratio was 10:1.

FIG. 5A: PMC159, A1847 target cell; FIG. 5B: PMC159, BxPC3 target cell; FIG. 5C: PMC159, Hela-CD19 target cell; FIG. 5D: PMC159, SKOV target cell; FIG. 5E: PMC804, A431 target cell.

FIGS. 6A-6B. The cytotoxic activity of Avelumab PD-L1 scFv-CAR-T cells against target cancer cells (6A: BxPC3 cells, 6B: SKOV-3 cells). Effector to target cells ratio was equal to 10:1.

FIGS. 7A-7B. Combination of CD24-CAR-T cells and Promab's PD-L1-CAR-T cells against cancer cells. 100% killing was observed in BxPC3 cells and >90% was observed in SKOV-3 cells with the combination.

### DETAILED DESCRIPTION OF THE INVENTION

### Definitions

As used herein, a “chimeric antigen receptor (CAR)” is a receptor protein that has been engineered to give T cells the new ability to target a specific protein. The receptor is chimeric because they combine both antigen-binding and T-cell activating functions into a single receptor. CAR is a fused protein comprising an extracellular domain capable of binding to an antigen, a transmembrane domain, and at least one intracellular domain. The “chimeric antigen receptor (CAR)” is sometimes called a “chimeric receptor”, a “T-body”, or a “chimeric immune receptor (CIR).”

The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell.

As used herein, a “domain” means one region in a polypeptide which is folded into a particular structure independently of other regions.

As used herein, a FLAG-tag, or FLAG octapeptide, or FLAG epitope, is a polypeptide protein tag that can be added to a protein using recombinant DNA technology, having the sequence motif DYKDDDDK (SEQ ID NO: 1). It can be fused to the C-terminus or the N-terminus of a protein, or inserted within a protein.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for engineering an scFv are known to a person skilled in the art.

As used herein, a “tumor antigen” means a biological molecule having antigenicity, expression of which causes cancer.

The inventors have generated mouse anti-human monoclonal antibody specifically targeting PD-L1 (Promab anti-PD-L1). The monoclonal anti-human PD-L1 antibody was generated against purified recombinant fragment of human PD-L1.

The inventors have produced PD-L1-CAR-T cells to target cancer cells overexpressing PD-L1 tumor antigen. The PD-L1-CAR-T cells of the present invention have high cytotoxic activity against several cancer cell lines and anti-tumor activity in vivo.

The present invention is directed to anti-human PD-L1 antibody or an antigen-binding fragment thereof, comprising VH having the amino acid of SEQ ID NO: 3 and VL having the amino acid of SEQ ID NO: 5. Antigen-binding fragments include Fab monomer, or Fab dimer (Fab′)₂, or scFv. In one embodiment, the monoclonal anti-human PD-L1 antibody is a single-chain variable fragment (scFv).

The present invention is also directed to a chimeric antigen receptor fusion protein comprising from N-terminus to C-terminus: (i) a single-chain variable fragment (scFv) against PD-L1, (ii) a transmembrane domain, (iii) at least one co-stimulatory domains, and (iv) an activating domain.

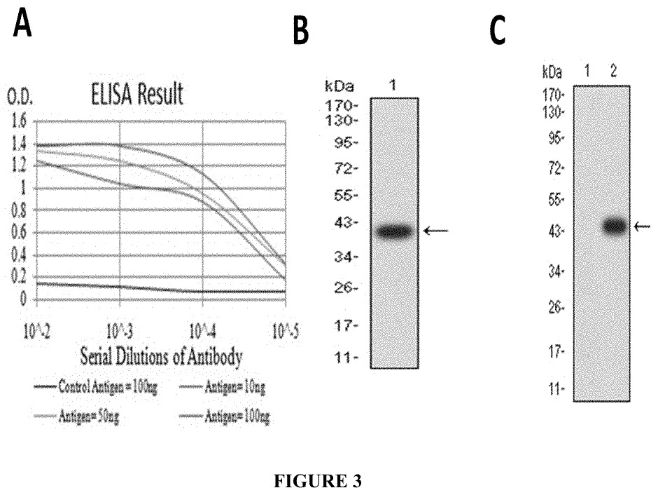

FIG. 2 shows two structures of PD-L1 CAR construct. The second-generation CAR is shown with CD28 (top panel) or 4-1BB (bottom panel) as a co-stimulatory domain. Abbreviations: Flag—FLAG tag; TM—transmembrane.

In the PD-L1 CAR construct, ScFv can be VH-linker-VL or VL-linker-VH.

In one embodiment, the co-stimulatory domain is selected from the group consisting of CD28, 4-1BB, GITR, ICOS-1, CD27, OX-40 and DAP10. A preferred the co-stimulatory domain is CD28.

A preferred activating domain is CD3 zeta (CD3 Z or CD3ζ).

The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, a CD3 zeta chain, CD28, CD3ε, CD45, CD4, CD5, CD8, CD9, CD16, CD22, CD33, CD37, CD64, CD80, CD86, CD134, CD137, ICOS, CD154, or a GITR can be used. The artificially designed transmembrane domain is a polypeptide mainly comprising hydrophobic residues such as leucine and valine. It is preferable that a triplet of phenylalanine, tryptophan and valine is found at each end of the synthetic transmembrane domain. Optionally, a short oligopeptide linker or a polypeptide linker, for example, a linker having a length of 2 to 10 amino acids can be arranged between the transmembrane domain and the intracellular domain. In one embodiment, a linker sequence having a glycine-serine continuous sequence can be used.

The present invention provides a nucleic acid encoding the PD-L1 CARs. The nucleic acid encoding the CAR can be prepared from an amino acid sequence of the specified CAR by a conventional method. A base sequence encoding an amino acid sequence can be obtained from the NCBI RefSeq IDs or accession numbers of GenBank for an amino acid sequence of each domain, and the nucleic acid of the present invention can be prepared using a standard molecular biological and/or chemical procedure. For example, based on the base sequence, a nucleic acid can be synthesized, and the nucleic acid of the present invention can be prepared by combining DNA fragments which are obtained from a cDNA library using a polymerase chain reaction (PCR).

A nucleic acid encoding the CAR of the present invention can be inserted into a vector, and the vector can be introduced into a cell. For example, a virus vector such as a retrovirus vector (including an oncoretrovirus vector, a lentivirus vector, and a pseudo type vector), an adenovirus vector, an adeno-associated virus (AAV) vector, a simian virus vector, a vaccinia virus vector or a Sendai virus vector, an Epstein-Barr virus (EBV) vector, and a HSV vector can be used. A virus vector lacking the replicating ability so as not to self-replicate in an infected cell is preferably used.

For example, when a retrovirus vector is used, a suitable packaging cell based on a LTR sequence and a packaging signal sequence possessed by the vector can be selected for preparing a retrovirus particle using the packaging cell. Examples of the packaging cell include PG13 (ATCC CRL-10686), PA317 (ATCC CRL-9078), GP+E-86 and GP+envAm-12, and Psi-Crip. A retrovirus particle can also be prepared using a 293 cell or a 293T cell having high transfection efficiency. Many kinds of retrovirus vectors produced based on retroviruses and packaging cells that can be used for packaging of the retrovirus vectors are widely commercially available from many companies.

A CAR-T cell binds to a specific antigen via the CAR, thereby a signal is transmitted into the cell, and as a result, the cell is activated. The activation of the cell expressing the CAR is varied depending on the kind of a host cell and an intracellular domain of the CAR, and can be confirmed based on, for example, release of a cytokine, improvement of a cell proliferation rate, change in a cell surface molecule, or the like as an index. For example, release of a cytotoxic cytokine (a tumor necrosis factor, lymphotoxin, etc.) from the activated cell causes destruction of a target cell expressing an antigen. In addition, release of a cytokine or change in a cell surface molecule stimulates other immune cells, for example, a B cell, a dendritic cell, a NK cell, and a macrophage.

The cell expressing the CAR can be used as a therapeutic agent for a disease. The therapeutic agent comprises the cell expressing the CAR as an active ingredient, and it may further comprise a suitable excipient.

The inventors have generated PD-L1-CAR-T (PD-L1-CAR-T) cells against different cancer cells overexpressing PD-L1 (ovarian, pancreatic and other cancers). The inventors have provided data demonstrating efficient expression of PD-L1 in different types of cancer (ovarian, pancreatic, and others). PD-L1-CAR-T cells express higher cytotoxic activity against PL-1-positive cancer cells than non-transduced T cells and Mock-CAR-T cells. In one embodiment, a FLAG tag added to the C-terminus of ScFv, which allows detection of CAR in the cells. The PD-L1 antibody is highly potent as a therapeutic agent in many clinical applications.

The advantage of the PD-L1 monoclonal antibody or PD-L1-ScFv of the present invention over known anti-PD-L1 antibodies such as Avelumab is that the present antibody is highly specific against PD-L1-positive cancer cells (ovarian, pancreatic and others). In addition, activity of Promab PD-L1-CAR-T is higher than that of Avelumab-PD-L1-CAR-T cells. The inventors have shown that Promab PD-L1 CAR-T cells are more effective than Avelumab PD-L1 CAR-T cells in killing several cancer cell lines.

The present monoclonal mouse anti-human PD-L1 antibody detects PD-L1 in PD-L1-positive cancer cells.

The present PD-L1 antibody can be used for immunotherapy applications: toxin/drug-conjugated Ab, monoclonal therapeutic antibody, humanization of PD-L1 antibody, CAR-T cell immunotherapy.

PD-L1-CAR-T cells using the present PD-L1 antibody can be effectively used to target PD-L1 antigen in PD-L1-positive cell lines such as ovarian, pancreatic, and cervical cancer.

PD-L1-CAR-T can be used in combination with different chemotherapy: checkpoint inhibitors; targeted therapies, small molecule inhibitors, and antibodies. PD-L1 antibody can be modified with site-directed mutagenesis for affinity tuning; it can be used for humanization and for complete human antibody generation.

PD-L1-CAR-T cells can be used clinically for targeting PD-L1-positive cells.

Modifications of co-activation domains: CD28, 4-1BB and others can be used to increase its efficacy. Tag-conjugated PD-L1 scFv can be used for CAR generation Third generation CAR-T or other co-activation signaling domains can be used for the same PD-L1-scFv inside CAR.

Combination of PD-L1 with other CAR targeting other tumor antigens or tumor microenvironment (VEGFR-1-3), or bispecific scFv-CAR can be used to enhance activity of monotherapy PD-L1-CAR.

Bi-specific antibodies with PD-L1 and CD3 or other antigens can be generated for therapy.

PD-L1 scFV, PD-L1 antibody, or PD-L1 CAR-T cells can be used together with another CAR to increase its activity. Dual targeting PD-L1 and another tumor antigen can enhance therapy. Also, co-transfection of PD-L1-CAR-T cells with other CAR-T cells can be used with to inhibit checkpoint signaling and to increase activity of CAR-T cells. The combination of CD24-CAR-T and PD-L1 CAR-T showed similar killing activity in cytotoxicity assay on two different cell lines. This can be applied for co-inhibition of two pathways in vivo, when PD-L1 pathway is activated in tumor microenvironment. In addition, bi-specific CAR-T cells with two scFv bound with a linker can be used for enhancing efficacy of single scFv CAR-T cells.

PD-L1 monoclonal antibody can be used as a single agent or in combination with other therapies. This combination therapy approach will increase efficacy of CAR-T.

PD-L1-CAR-T cells can be used against cancer stem cells that are most resistant against chemotherapy and form aggressive tumors.

PD-L1-CAR can be used for generating other types of cells such as CAR-natural killer (NK) cells, iPS (induced pluripotent)-NK or iPS-T cells, macrophages, gamma-delta T cells and other hematopoietic cells, which can target PD-L1-positive cancers. The present invention provides T cells, or NK cells, or macrophages, or hematopoietic cells, modified to express the PD-L1-CAR.

The following examples further illustrate the present invention. These examples are intended merely to be illustrative of the present invention and are not to be construed as being limiting.

### EXAMPLES

### Example 1. PD-L1 Antibody and Activity

We generated mouse monoclonal anti-human PD-L1 antibody using hybridoma (clone 7D2A10). The hybridoma was generated against purified recombinant fragment (24-153 amino acid sequence) of human PD-L1: expressed in E. coli. The hybridoma technology is standard and described in [4]. The antibody detects PD-L1 extracellular domain and is IgG2b type.

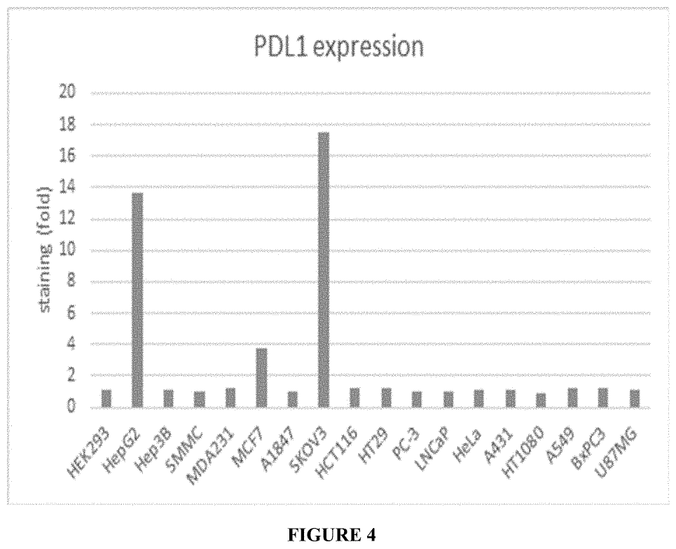

FIG. 3 shows that this anti-human PD-L1 antibody detects PD-L1 antigen by ELISA and by Western blotting. In FIG. 3A, the ELISA result with Control Antigen (100 ng), PD-L1 Antigen (10 ng), PDL-1 Antigen (50 ng), PD-L1 Antigen (100 ng) were shown from bottom to top. The ELISA showed no binding with Control Antigen (100 ng), and dose-dependent increased binding with PD-L1 Antigen (10 ng), PD-L1 Antigen (50 ng), and PD-L1 Antigen (100 ng). In FIG. 3B, Western blot analysis showed the binding of this antibody to PD-L1 extracellular domain (AA: 24-153, expected MW of 40.1 kDa). In FIG. 3C, Western blot analysis showed absence of binding of this antibody to PD-L1 in (1) HEK293 cell lysate, and presence of binding in (2) HEK 293 cell lysate transfected with PD-L1 extracellular domain fused to human Fc (PD-L1-hFc).

This PD-L1 antibody detects high level of PD-L1 in tumor tissues and in several cancer cell lines by flow cytometry.

This antibody detected moderate PD-L1 expression in liver cancer and some binding in normal liver, lung, uterus and hypohysis. Most normal tissues (colon, duodenum, rectum, testicle, esophagus, brain, muscle, pancreas, kidney, stomach, prostate, tonsil, and spleen) showed negative PD-L1 expression by this antibody.

The absence of staining in most normal tissues is advantageous for using this antibody in CAR-T format due to less off-target and off tumor activity.

### Example 2. PD-L1 VH, VL and scFv Sequences

We sequenced anti-PD-L1 antibody from a hybridoma clone (#7D2A10), positive for binding with PD-L1 antigen by ELISA. The structure of anti-PD-L1 scFv is: VH-linker-VL.

PD-L1 VH Nucleotide Sequence
(SEQ ID NO: 2)
CAGATCCAGTTGGTGCAGTCTGGACCTGAGCTGAAGAACCCTGGAGAGAC
AGTCAAGATCTCCTGCAAGGCTTCTGGGTATACCTTCACAAACTATGGAA
TGAACTGGGTGAAGCAGGCTCCAGGAAAGGGTTTAAAGTGGATGGGGTGG
ATAAACACCCACACTGGAGAGCCAACATATGCTGATGACTTCAAGGGACG
GTTTGCCTTCTCTTCGGAAACCTCTGCCAGCTCTGCCTATTTGCAGATCA
ACAACCTCAAAAATGATGACATGGCTACATATTTCTGTGCAAAAGGTACC
CACAGAGAAGAAATTCCGGCCTGGTTCGCTTACTGGGGCCAAGGGACTCT
GGTCACTGTCTCTGCA
PD-L1 VH Amino Acid Sequence
(SEQ ID NO: 3)
Q I Q L V Q S G P E L K N P G E T V K I S C K A S
G Y T F T N Y G M N W V K Q A P G K G L K W M G W
I N T H T G E P T Y A D D F K G R F A F S S E T S
A S S A Y L Q I N N L K N D D M A T Y F C A K G T
H R E E I P A W F A Y W G Q G T L V T V S A
PD-L1 VL Nucleotide Sequence
(SEQ ID NO: 4)
GATGTTTTGATGACCCAAACTCCACTCTCCCTGCCTGTCAGTCTTGGAGA
TCAAGCCTCCATCTCTTGCAGATCTAGTCAGAGCATTGTACATAGTAATG
GAAACACCTATTTAGAATGGTACCTGCAGAAACCAGGCCAGTCTCCAGAG
CTCCTGATCTACAAAGTTTCCAACCTATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTACTGCTTTCAAGGTTCACATGTTCCT
CCCACGTTCGGTGCTGGGACCAAGCTGGAGCTGAAACGG
PD-L1 VL Amino Acid Sequence
(SEQ ID NO: 5)
D V L M T Q T P L S L P V S L G D Q A S I S C R S
S Q S I V H S N G N T Y L E W Y L Q K P G Q S P E
L L I Y K V S N L F S G V P D R F S G S G S G T D
F T L K I S R V E A E D L G V Y Y C F Q G S H V P
P T F G A G T K L E L K R
Linker Nucleotide Sequence
(SEQ ID NO: 6)
GGTGGCGGTGGTTCT GGTGGCGGTGGTTCT GGTGGCGGTGGTTCT
Linker Amino Acid Sequence
(SEQ ID NO: 7)
GGGGS GGGGS GGGGS

PD-L1 scFv Nucleotide Sequence: (SEQ ID NO: 8)
The bold, larger font highlights the nucleotide sequence of V_{H}; the underlined highlights the nucleotide sequence of V_{L}; in between (italicized) is the nucleotide sequence encoding a linker.

CAGATCCAGTTGGTGCAGTCTGGACCTGAGCTGAAGAACCCTGGAGAGAC
AGTCAAGATCTCCTGCAAGGCTTCTGGGTATACCTTCACAAACTATGGAA
TGAACTGGGTGAAGCAGGCTCCAGGAAAGGGTTTAAAGTGGATGGGGTGG
ATAAACACCCACACTGGAGAGCCAACATATGCTGATGACTTCAAGGGACG
GTTTGCCTTCTCTTCGGAAACCTCTGCCAGCTCTGCCTATTTGCAGATCA
ACAACCTCAAAAATGATGACATGGCTACATATTTCTGTGCAAAAGGTACC
CACAGAGAAGAAATTCCGGCCTGGTTCGCTTACTGGGGCCAAGGGACTCT
GGTCACTGTCTCTGCA GGTGGCGGTGGTTCT GGTGGCGGTGGTTCT
GGTGGCGGTGGTTCT GATGTTTTGATGACCCAAACTCCACTCTCCCTGC
CTGTCAGTCTTGGAGATCAAGCCTCCATCTCTTGCAGATCTAGTCAGAGC
ATTGTACATAGTAATGGAAACACCTATTTAGAATGGTACCTGCAGAAACC
AGGCCAGTCTCCAGAGCTCCTGATCTACAAAGTTTCCAACCTATTTTCTG
GGGTCCCAGACAGGTTCAGTGGCAGTGGATCAGGGACAGATTTCACACTC
AAGATCAGCAGAGTGGAGGCTGAGGATCTGGGAGTTTATTACTGCTTTCA
AGGTTCACATGTTCCTCCCACGTTCGGTGCTGGGACCAAGCTGGAGCTGA
AACGG
PD-L1 ScFv Amino-acid Sequence
(SEQ ID NO: 9)
Q I Q L V Q S G P E L K N P G E T V K I S C K A S
G Y T F T N Y G M N W V K Q A P G K G L K W M G W
I N T H T G E P T Y A D D F K G R F A F S S E T S
A S S A Y L Q I N N L K N D D M A T Y F C A K G T
H R E E I P A W F A Y W G Q G T L V T V S A G G G
G S G G G G S G G G G S D V L M T Q T P L S L P V
S L G D Q A S I S C R S S Q S I V H S N G N T Y L
E W Y L Q K P G Q S P E L L I Y K V S N L F S G V
P D R F S G S G S G T D F T L K I S R V E A E D L
G V Y Y C F Q G S H V P P T F G A G T K L E L K R

### Example 3. PD-L1 Lentiviral CAR Construct

The inventors generated PD-L1 CAR constructs inside lentiviral vector cloned into Xba I and Eco R I sites of lentiviral vector. pCD510-FMC63-28z lentiviral CAR construct contained the PD-L1 ScFv-Flag tag-CD8 hinge, CD28 transmembrane/activation-CD3 zeta insert between the Xba I and EcoR I cloning sites under CMV promoter (PMC159). Flag tag was inserted for easier detection of CAR-positive T cells. The inventors also generated PMC804 CAR with same PD-L1-CAR scfv with no FLAG tag after scfv containing 41BB costimulatory domain instead of CD28 and regulated by MNDU3 promoter for higher expression of CAR.

The lentiviruses were generated in 293T cells and titer was established by RT-PCR. Then equal dose of lentiviruses was used for transduction of T cells.

### Example 4A. PD-L1 CAR with CD28 as a Co-Stimulating Domain (PMC159)

The scheme of PD-L1-CAR construct is shown on FIG. 2. Lentiviral vector Lenti CMV-MCS-EF1a-puro, was used for cloning of all scFv CAR sequences.

The following nucleotide and amino acid sequences show PD-L1 ScFv Flag-CD8 hinge-TM28-CD28-CD3 zeta of the present invention. The structure includes human CD8 signaling peptide (CD8 leader), PD-L1 scFv (V_{H}-Linker 3×(G4S)-V_{L}), FLAG, CD8 hinge, CD28 transmembrane, activation domains, CD3 zeta (FIG. 2).

<CD8 leader>
Nucleotide Sequence
(SEQ ID NO: 10)
ATGGCCTTACCAGTGACCGCCTTGCTCCTGCCGCTGGCCTTGCTGCTCCA
CGCCGCCAGGCCG
Amino-acid sequence
(SEQ ID NO: 11)
MALPVTALLLPLALLLHAARP
<Nhe restriction I site>
GCTAGC
Amino acid sequence
<AS>
<PD-L1 scFV>
See Example 2, SEQ ID NOs: 8 and 9.
<FLAG>
Nucleotide Sequence
(SEQ ID NO: 12)
GACTACAAAGACGATGACGACAAG
Amino Acid Sequence
(SEQ ID NO: 1)
DYKDDDDK
<XhoI restriction site>
Nucleotide Sequence
CTCGAG
Amino acid Sequence
LE
<CD8 hinge>
Nucleotide Sequence
(SEQ ID NO: 13)
AAGCCCACCACGACGCCAGCGCCGCGACCACCAACACCGGCGCCCACCAT
CGCGTCGCAGCCCCTGTCCCTGCGCCCAGAGGCGAGCCGGCCAGCGGCGG
GGGGCGCAGTGCACACGAGGGGGCTGGACTTCGCCAGTGAT
Amino Acid sequence
(SEQ ID NO: 14)
KPTTTPAPRPPTPAPTIASQPLSLRPEASRPAAGGAVHRGLDFASD
<Spacer>
Nucleotide sequence
aagccc
Amino Acid Sequence
KP
<CD28 TM/co-stimulating>
Nucleotide Sequence
(SEQ ID NO: 15)
TTTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGGC
TCCTGCACAGTGACTACATGAACATGACTCCCCGCCGCCCCGGGCCCACC
CGCAAGCATTACCAGCCCTATGCCCCACCACGCGACTTCGCAGCCTATCG
CTCC
Amino Acid sequence
(SEQ ID NO: 16)
FWVLVVVGGVLACYSLLVTVAFIIFWV/RSKRSRLLHSDYMNMTPRRPGP
TRKHYQPYAPPRDFAAYRS
<CD3 zeta>
Nucleotide Sequence
(SEQ ID NO: 17)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGG
GCAAGGGGCACGATGGCCTTTACCAGGGTCTCAGTACAGCCACCAAGGAC
ACCTACGACGCCCTTCACATGCAGGCCCTGCCCCCTCGCTAA
Amino Acid sequence
(SEQ ID NO: 18)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR
Nucleotide sequence of PD-L1-CAR (PMC 159, FLAG is
underlined),
SEQ ID NO: 19
ATGGCCTTACCAGTGACCGCCTTGCTCCTGCCGCTGGCCTTGCTGCTCCA
CGCCGCCAGGCCGgctagcCAGATCCAGTTGGTGCAGTCTGGACCTGAGC
TGAAGAACCCTGGAGAGACAGTCAAGATCTCCTGCAAGGCTTCTGGGTAT
ACCTTCACAAACTATGGAATGAACTGGGTGAAGCAGGCTCCAGGAAAGGG
TTTAAAGTGGATGGGGTGGATAAACACCCACACTGGAGAGCCAACATATG
CTGATGACTTCAAGGGACGGTTTGCCTTCTCTTCGGAAACCTCTGCCAGC
TCTGCCTATTTGCAGATCAACAACCTCAAAAATGATGACATGGCTACATA
TTTCTGTGCAAAAGGTACCCACAGAGAAGAAATTCCGGCCTGGTTCGCTT
ACTGGGGCCAAGGGACTCTGGTCACTGTCTCTGCA
GGTGGCGGTGGTTCT GGTGGCGGTGGTTCT GGTGGCGGTGGTTCT
GATGTTTTGATGACCCAAACTCCACTCTCCCTGCCTGTCAGTCTTGGAGA
TCAAGCCTCCATCTCTTGCAGATCTAGTCAGAGCATTGTACATAGTAATG
GAAACACCTATTTAGAATGGTACCTGCAGAAACCAGGCCAGTCTCCAGAG
CTCCTGATCTACAAAGTTTCCAACCTATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTACTGCTTTCAAGGTTCACATGTTCCT
CCCACGTTCGGTGCTGGGACCAAGCTGGAGCTGAAACGG GACTACAAAG
ACGATGACGACAAGctcgagAAGCCCACCACGACGCCAGCGCCGCGACCA
CCAACACCGGCGCCCACCATCGCGTCGCAGCCCCTGTCCCTGCGCCCAGA
GGCGAGCCGGCCAGCGGCGGGGGGCGCAGTGCACGAGGGGGCTGGACTTC
GCCAGTGATaagcccttttgggtgctggtggtggttggtggagtcctggc
ttgctatagcttgctagtaacagtggcctttattattttctgggtgagga
gtaagaggagcaggctcctgcacagtgactacatgaacatgactccccgc
cgccccgggcccacccgcaagcattaccagccctatgcccaccacgcgac
ttcgcagcctatcgctccAGAGTGAAGTTCAGCAGGAGCGCAGACGCCCC
CGCGTACCAGCAGGGCCAGAACCAGCTCTATAACGAGCTCAATCTAGGAC
GAAGAGAGGAGTACGATGTTTTGGACAAGAGACGTGGCCGGGACCCTGAG
ATGGGGGGAAAGCCGCAGAGAAGGAAGAACCCTCAGGAAGGCCTGTACAA
TGAACTGCAGAAAGATAAGATGGCGGAGGCCTACAGTGAGATTGGGATGA
AAGGCGAGCGCCGGAGGGGCAAGGGGCACGATGGCCTTTACCAGGGTCTC
AGTACAGCCACCAAGGACACCTACGACGCCCTTCACATGCAGGCCCTGCC
CCCTCGCTAA
Amino Acid sequence of PD-L1-CAR protein, PMC159,
SEQ ID NO: 20
M A L P V T A L L L P L A L L L H A A R P A S Q I
Q L V Q S G P E L K N P G E T V K I S C K A S G Y
T F T N Y G M N W V K Q A P G K G L K W M G W I N
T H T G E P T Y A D D F K G R F A F S S E T S A S
S A Y L Q I N N L K N D D M A T Y F C A K G T H R
E E I P A W F A Y W G Q G T L V T V S A G G G G S
G G G G S G G G G S D V L M T Q T P L S L P V S L
G D Q A S I S C R S S Q S I V H S N G N T Y L E W
Y L Q K P G Q S P E L L I Y K V S N L F S G V P D
R F S G S G S G T D F T L K I S R V E A E D L G V
Y Y C F Q G S H V P P T F G A G T K L E L K R D Y
K D D D D K L E K P T T T P A P R P P T P A P T I
A S Q P L S L R P E A S R P A A G G A V H T R G L
D F A S D K P F W V L V V V G G V L A C Y S L L V
T V A F I I F W V R S K R S R L L H S D Y M N M T
P R R P G P T R K H Y Q P Y A P P R D F A A Y R S
R V K F S R S A D A P A Y Q Q G Q N Q L Y N E L N
L G R R E E Y D V L D K R R G R D P E M G G K P Q
R R K N P Q E G L Y N E L Q K D K M A E A Y S E I
G M K G E R R R G K G H D G L Y Q G L S T A T K D
T Y D A L H M Q A L P P

### Example 4B. PD-L1 CAR with 4-1BB as a Co-Stimulating Domain (PMC804)

The nucleotide and amino acid sequences of this CAR are the same to those of Example 4A except this CAR does not have FLAG tag and it replaces CD28 with 4-1BB as a co-stimulating domain.

<41BB domain/co-stimulating
Nucleotide Sequence,
SEQ ID NO: 21
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG
Amino Acid Sequence,
SEQ ID NO: 22
KRGRKKLLYIFKQPFMRPVQTTQEEDGCSCRFPEEEEGGCEL
Nucleotide sequence of PD-L1-CAR
(PMC 804, 4-1BB underlined),
SEQ ID NO: 23
ATGGCCTTACCAGTGACCGCCTTGCTCCTGCCGCTGGCCTTGCTGCTCCA
CGCCGCCAGGCCGgctagcCAGATCCAGTTGGTGCAGTCTGGACCTGAGC
TGAAGAACCCTGGAGAGACAGTCAAGATCTCCTGCAAGGCTTCTGGGTAT
ACCTTCACAAACTATGGAATGAACTGGGTGAAGCAGGCTCCAGGAAAGGG
TTTAAAGTGGATGGGGTGGATAAACACCCACACTGGAGAGCCAACATATG
CTGATGACTTCAAGGGACGGTTTGCCTTCTCTTCGGAAACCTCTGCCAGC
TCTGCCTATTTGCAGATCAACAACCTCAAAAATGATGACATGGCTACATA
TTTCTGTGCAAAAGGTACCCACAGAGAAGAAATTCCGGCCTGGTTCGCTT
ACTGGGGCCAAGGGACTCTGGTCACTGTCTCTGCAGGTGGCGGTGGTTCT
GGTGGCGGTGGTTCTGGTGGCGGTGGTTCTGATGTTTTGATGACCCAAAC
TCCACTCTCCCTGCCTGTCAGTCTTGGAGATCAAGCCTCCATCTCTTGCA
GATCTAGTCAGAGCATTGTACATAGTAATGGAAACACCTATTTAGAATGG
TACCTGCAGAAACCAGGCCAGTCTCCAGAGCTCCTGATCTACAAAGTTTC
CAACCTATTTTCTGGGGTCCCAGACAGGTTCAGTGGCAGTGGATCAGGGA
CAGATTTCACACTCAAGATCAGCAGAGTGGAGGCTGAGGATCTGGGAGTT
TATTACTGCTTTCAAGGTTCACATGTTCCTCCCACGTTCGGTGCTGGGAC
CAAGCTGGAGCTGAAACGGctcgagAAGCCCACCACGACGCCAGCGCCGC
GACCACCAACACCGGCGCCCACCATCGCGTCGCAGCCCCTGTCCCTGCGC
CCAGAGGCGAGCCGGCCAGCGGCGGGGGGCGCAGTGCACACGAGGGGGCT
GGACTTCGCCAGTGATaagcccttttgggtgctggtggtggttggtggag
tcctggcttgctatagcttgctagtaacagtggcctttattattttctgg
gtgAAACGGGGCAGAAAGAAACTCCTGTATATATTCAAACAACCATTTAT
GAGACCAGTACAAACTACTCAAGAGGAAGATGGCTGTAGCTGCCGATTTC
CAGAAGAAGAAGAAGGAGGATGTGAACTGAGAGTGAAGTTCAGCAGGAGC
GCAGACGCCCCCGCGTACCAGCAGGGCCAGAACCAGCTCTATAACGAGCT
CAATCTAGGACGAAGAGAGGAGTACGATGTTTTGGACAAGAGACGTGGCC
GGGACCCTGAGATGGGGGGAAAGCCGCAGAGAAGGAAGAACCCTCAGGAA
GGCCTGTACAATGAACTGCAGAAAGATAAGATGGCGGAGGCCTACAGTGA
GATTGGGATGAAAGGCGAGCGCCGGAGGGGCAAGGGGCACGATGGCCTTT
ACCAGGGTCTCAGTACAGCCACCAAGGACACCTACGACGCCCTTCACATG
CAGGCCCTGCCCCCTCGCTAA
Amino Acid sequence of PD-L1-CAR (PMC 804, 4-1BB
underlined),
SEQ ID NO: 24
MALPVTALLLPLALLLHAARPASQIQLVQSGPELKNPGETVKISCKASGY
TFTNYGMNWVKQAPGKGLKWMGWINTHTGEPTYADDFKGRFAFSSETSAS
SAYLQINNLKNDDMATYFCAKGTHREEIPAWFAYWGQGTLVTVSAGGGGS
GGGGSGGGGSDVLMTQTPLSLPVSLGDQASISCRSSQSIVHSNGNTYLEW
YLQKPGQSPELLIYKVSNLFSGVPDRFSGSGSGTDFTLKISRVEAEDLGV
YYCFQGSHVPPTFGAGTKLELKRLEKPTTTPAPRPPTPAPTIASQPLSLR
PEASRPAAGGAVHTRGLDFASDKPFWVLVVVGGVLACYSLLVTVAFIIFW
VKRGRKKLLYIFKOPFMRPVOTTQEEDGCSCRFPEEEEGGCELRVKFSRS
ADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHM
QALPPR

### Example 5. CAR Lentivirus Production

DNAs encoding PD-L1 scFv were synthesized and subcloned into a third-generation lentiviral vector, Lenti CMV-MCS-EF1a-puro by Syno Biological (Beijing, China). The lentiviral constructs were sequenced in both directions to confirm the construct sequence and used for lentivirus production. Ten million growth-arrested HEK293FT cells (Thermo Fisher) were seeded into T75 flasks and cultured overnight, then transfected with the pPACKH1 Lentivector Packaging mix (System Biosciences, Palo Alto, Calif.) and 10 μg of each lentiviral vector using the CalPhos Transfection Kit (Takara, Mountain View, Calif.). The next day the medium was replaced with fresh medium, and 48 hour later the lentivirus-containing medium was collected. The medium was cleared of cell debris by centrifugation at 2100 g for 30 min. The virus particles were collected by centrifugation at 112,000 g for 100 min, suspended in DMEM or AIM V medium, aliquoted and frozen at −80° C. The titers of the virus preparations were determined by quantitative RT-PCR using the Lenti-X qRT-PCR kit (Takara) according to the manufacturer's protocol and the 7900HT thermal cycler (Thermo Fisher). The lentiviral titers were >1×10⁸ pfu/ml.

### Example 6. Peripheral Blood Mononuclear Cell (PBMC) Isolation from Whole Blood

Whole blood (Stanford Hospital Blood Center, Stanford, Calif.) was collected from an individual or from mixed donors (depending on the amount of blood required) in 10 mL fractions and isolated using Ficol-Paque PLUS. The layer of cells containing peripheral blood mononuclear cells (PBMC), seen at the diluted plasma/Ficoll interface was removed and avoiding any Ficoll. To ensure complete removal of Ficoll, platelets, and plasma proteins, PBMC's were washed twice with PBS for a total volume of 40 ml, and centrifuge at 200×g for 10 min at room temperature. Cells were then counted with a hemocytomter. If washed PBMC's were to be used immediately, they were washed once with CAR-T media (AIM V-AlbuMAX(BSA)(Life Technologies), with 5% AB serum and 1.25 ug/mL amphotericin B (Gemini Bioproducts, Woodland, Calif.), 100 U/mL penicillin, and 100 ug/mL streptomycin). If PBMC's were to be frozen, the washed cells were resuspended in transfer insulated vials to −80° C. for 24 hr, before storing in liquid nitrogen.

### Example 7. T-Cell Activation from PBMC

The PBMC isolated cells were cultivated in CAR-T media (AIM V-AlbuMAX(BSA, Life Technologies), with 5% AB serum and 1.25 μg/mL amphotericin B (Gemini Bioproducts, Woodland, Calif.), 100 U/mL penicillin, and 100 μg/mL streptomycin), in the presence of human interleukin-2 300 U/mL (huIL-2, Invitrogen), at a concentration of 5×10⁵ cells/mL The T were activated with CD3/CD28 beads and incubated at 37° C. in the presence of CO₂ for 24 hr before CAR viral transduction.

### Example 8. T-Cell Transduction and Expansion

Following activation of PBMC, cells were incubated for 24 hr at 37° C., 5% CO₂. To each well of 1×10⁶ cells, 5×10⁶ lentivirus and 2 μL/mL of media of Transplus (Alstem, Richmond, Calif.) (a final dilution of 1:500) were added. Cells were incubated for an additional 24 hr before repeating addition of virus. Cells were then grown in the continued presence of 300 U/Ml of IL-2 for a period of 12-14 days. Cells concentrations were analyzed every 2-3 days, with media being added at that time to dilute the cell suspension to 1×10⁶ cells/mL. The CAR expression can be verified with FLAG antibody or with anti-mouse F(ab)₂ detecting Flag-tagged scFv or untagged scFv expression.

### Example 9. Cytotoxicity Assay

The cytotoxicity was performed using ACEA machine according to manufacturer's protocol listed below.

Adherent target cancer cells were seeded into 96-well E-plates (Acea Biosciences, San Diego, Calif.) at 1×10⁴ cells per well and monitored in culture overnight with the impedance-based real-time cell analysis (RTCA) iCELLigence system (Acea Biosciences). The next day, the medium was removed and replaced with AIM V-AlbuMAX medium containing 10% FBS±1×10⁵ effector cells (CAR-T cells or non-transduced T cells), in triplicate. The cells in the E-plates were monitored for another 2-3 days with the RTCA system, and impedance was plotted over time. Cytolysis was calculated as (impedance of target cells without effector cells−impedance of target cells with effector CAR-T cells)×100/impedance of target cells without effector cells.

### Example 10. Expression of PD-L1 in Different Cancer and Normal Tissues

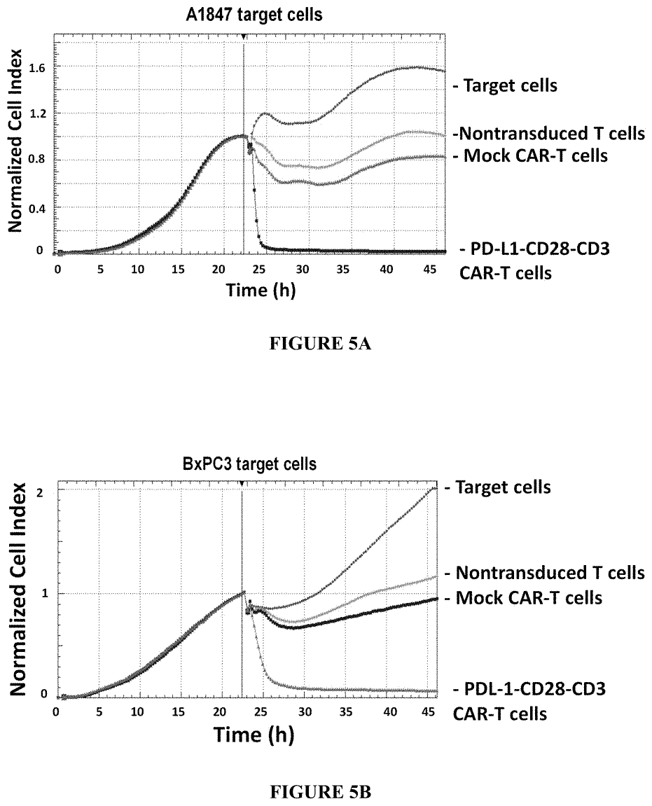

The staining with PD-L1 monoclonal antibody demonstrated high staining with several cancer cell lines: ovarian cancer SKOV-3, hepatocellular carcinoma, HepG2, and moderate with breast MCF-7 cell lines (FIG. 4). Normal HEK-293 cells, cancer cells: HT29, MDA-231, HCT116 and others were negative.

### Example 11. Promab PD-L1-CAR Expressed High Cytotoxic Activity Against PD-L1-Positive Cancer Cells

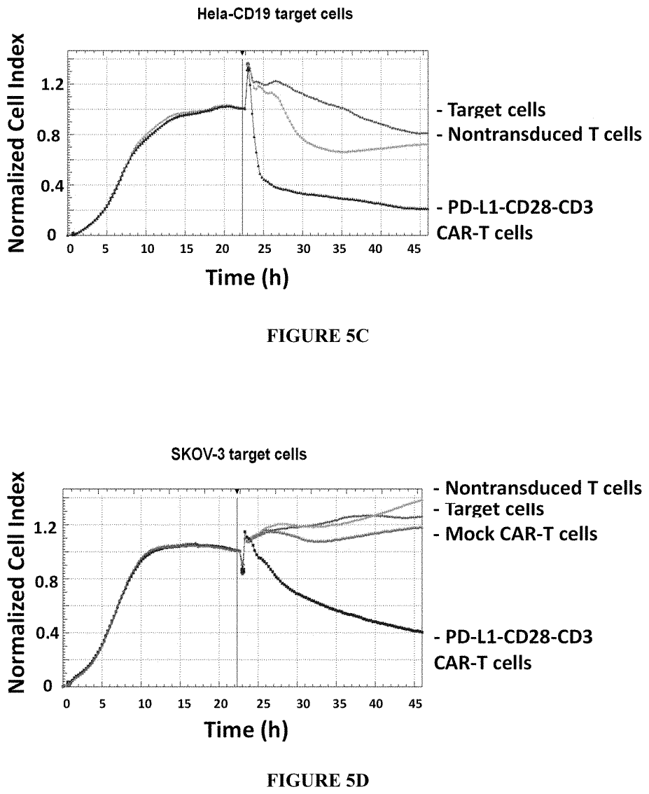

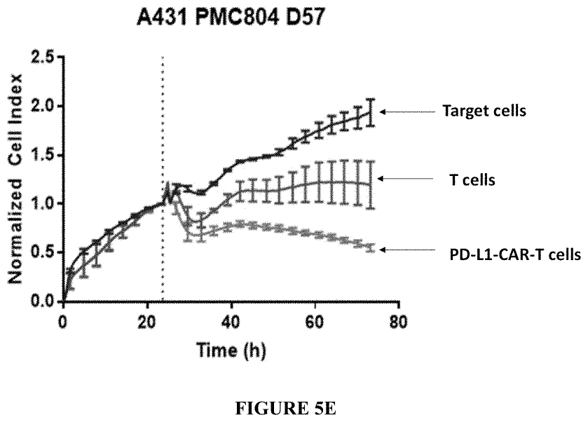

The real-time cytotoxicity assay demonstrated high cytotoxic activity of Promab PD-L1-CD28-CD3 CAR cells (PMC159) against high PD-L1-positive cancer cells: ovarian cancer A1847 cells, pancreatic cancer BxpC3 cells, cervical cancer Hela-CD19 cells, and ovarian cancer SKOV-3 (FIG. 5A-5D).

Promab PD-L1-CD28-CD3 CAR-T (PMC159) had 100% killing activity against ovarian cancer A1847 cells (FIG. 5A), almost 100% against pancreatic cancer BxPC3 cell lines (FIG. 5B), and >75% against cervical cancer Hela-CD19 cells (FIG. 5C).

We also tested PD-L1-41BB-CD3 CAR-T cells (PMC804) against A431 epidermal cancer cells, and found that they killed cancer cells (FIG. 5E). Thus, CAR-T cells with CD28 or 41BB costimulatory domain are active against cancer cells.

### Example 12. Comparison of CAR-T Cells with Promab's PD-L1 scFv Versus CAR-T Cells with Avelumab PD-L1-scFv

We compared Promab PD-L1 scFv with published PD-L1 scFv from antibody Avelumab that was approved by FDA to treat Merkel cell carcinoma. The sequence of published Avelumab PD-L1 scFv with no FLAG tag at the C-terminus was inserted into CAR. The general structure of Avelumab PD-L1CAR-T is shown in FIG. 2. The sequences of Avelumab PD-L1 scFv is shown below.

PD-L1 (Avelumab) VH, Nucleotide Sequence
(SEQ ID NO: 25)
gaagtgcagctgctggaaagcggcggcggcctggtgcagccgggcggcag
cctgcgcctgagctgcgcggcgagcggctttacctttagcagctatatta
tgatgtgggtgcgccaggcgccgggcaaaggcctggaatgggtgagcagc
atttatccgagcggcggcattaccttttatgcggataccgtgaaaggccg
ctttaccattagccgcgataacagcaaaaacaccctgtatctgcagatga
acagcctgcgcgcggaagataccgcggtgtattattgcgcgcgcattaaa
ctgggcaccgtgaccaccgtggattattggggccagggcaccctggtgac
cgtgagcagc
PD-L1 (Avelumab) VH, Amino Acid Sequence
(SEQ ID NO: 26)
EVQLLESGGG LVQPGGSLRL SCAASGFTFS SYIMMWVRQA
PGKGLEWVSS IYPSGGITFY ADTVKGRFTI SRDNSKNTLY
LQMNSLRAED TAVYYCARIK LGTVTTVDYW GQGTLVTVSS
Linker Nucleotide Sequence
(SEQ ID NO: 27)
ggcggcggcggcagcggcggcggcggcagcggcggcggcggcagc
Linker Amino Acid Sequence
(SEQ ID NO: 7)
GGGGSGGGGSGGGGS
PD-L1 (Avelumab) VL, Nucleotide Sequence
(SEQ ID NO: 28)
cagagcgcgctgacccagccggcgagcgtgagcggcagcccgggccagag
cattaccattagctgcaccggcaccagcagcgatgtgggcggctataact
atgtgagctggtatcagcagcatccgggcaaagcgccgaaactgatgatt
tatgatgtgagcaaccgcccgagcggcgtgagcaaccgctttagcggcag
caaaagcggcaacaccgcgagcctgaccattagcggcctgcaggcggaag
atgaagcggattattattgcagcagctataccagcagcagcacccgcgtg
tttggcaccggcaccaaagtgaccgtgctg
PD-L1 (Avelumab) VL, Amino Acid Sequence
(SEQ ID NO: 29)
Q S A L T Q P A S V S G S P G Q S I T I S C T G T
S S D V G G Y N Y V S W Y Q Q H P G K A P K L Met
I Y D V S N R P S G V S N R F S G S K S G N T A S
L T I S G L Q A E D E A D Y Y C S S Y T S S S T R
V F G T G T K V T V L
PD-L1 (Avelumab) ScFv Nucleotide Sequence
(SEQ ID NO: 30)
gaagtgcagctgctggaaagcggcggcggcctggtgcagccgggcggcag
cctgcgcctgagctgcgcggcgagcggctttacctttagcagctatatta
tgatgtgggtgcgccaggcgccgggcaaaggcctggaatgggtgagcagc
atttatccgagcggcggcattaccttttatgcggataccgtgaaaggccg
ctttaccattagccgcgataacagcaaaaacaccctgtatctgcagatga
acagcctgcgcgcggaagataccgcggtgtattattgcgcgcgcattaaa
ctgggcaccgtgaccaccgtggattattggggccagggcaccctggtgac
cgtgagcagcggcggcggcggcagcggcggcggcggcagcggcggcggcg
gcagccagagcgcgctgacccagccggcgagcgtgagcggcagcccgggc
cagagcattaccattagctgcaccggcaccagcagcgatgtgggcggcta
taactatgtgagctggtatcagcagcatccgggcaaagcgccgaaactga
tgatttatgatgtgagcaaccgcccgagcggcgtgagcaaccgctttagc
ggcagcaaaagcggcaacaccgcgagcctgaccattagcggcctgcaggc
ggaagatgaagcggattattattgcagcagctataccagcagcagcaccc
gcgtgtttggcaccggcaccaaagtgaccgtgctg
PD-L1 (Avelumab) ScFv Amino Acid Sequence
(SEQ ID NO: 31)
E V Q L L E S G G G L V Q P G G S L R L S C A A S
G F T F S S Y I M M W V R Q A P G K G L E W V S S
I Y P S G G I T F Y A D T V K G R F T I S R D N S
K N T L Y L Q M N S L R A E D T A V Y Y C A R I K
L G T V T T V D Y W G Q G T L V T V S S G G G G S
G G G G S G G G G S Q S A L T Q P A S V S G S P G
Q S I T I S C T G T S S D V G G Y N Y V S W Y Q Q
H P G K A P K L M I Y D V S N R P S G V S N R F S
G S K S G N T A S L T I S G L Q A E D E A D Y Y C
S S Y T S S S T R V F G T G T K V T V L

Avelumab PD-L1 CAR-T cells were generated according to Example 5.

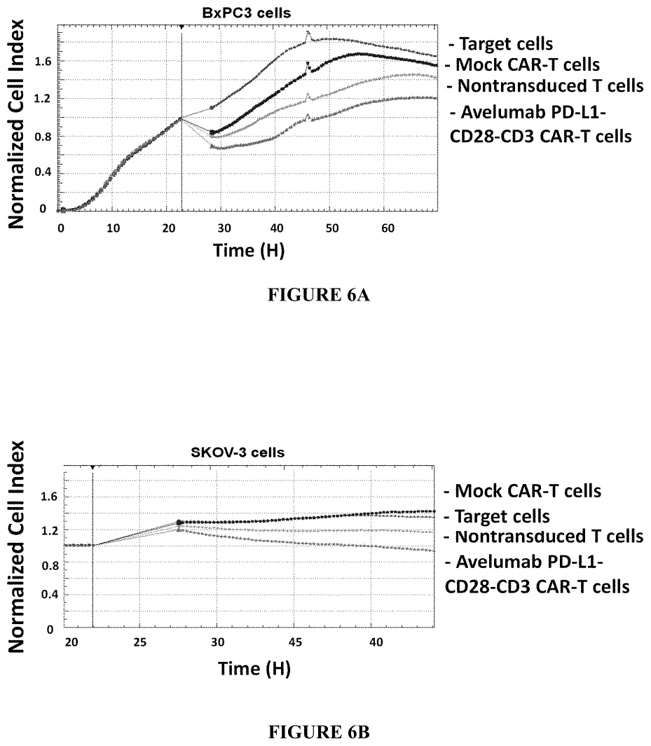

Avelumab PD-L1 CAR-T cells were used in cytotoxicity assays (FIG. 6). The results show that Promab PD-L1 CAR-T cells are more effective than Avelumab PD-L1 CAR-T cells in killing the same cancer cell lines. The Avelumab PD-L1 CAR-T cells had about 25% killing activity against BxPC3 cells, whereas PMC159 PD-L1 CAR-T cells had almost 100% killing activity against the same cells (see Example 11, FIG. 5B). The Avelumab PD-L1 CAR-T cells had about <35% killing activity against SKOV-3 cells, whereas PMC159 PD-L1 CAR-T cells had >>67% killing activity against the same cells (see Example 11, FIG. 5D).

### Example 13. Combination of CD24 and Promab PD-L1-CAR-T Cells Against Cancer Cells

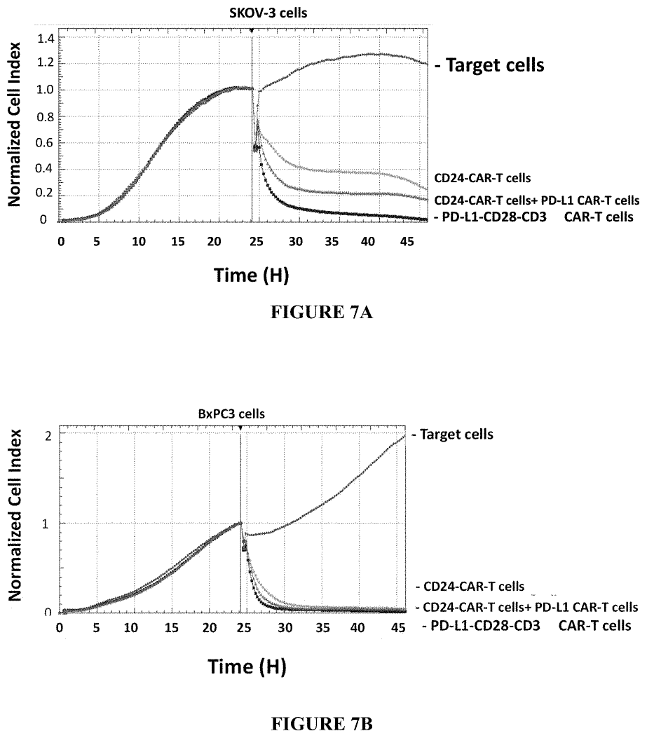

FIGS. 7A-7B shows the combination of CD24-CAR-T cells and PMC159 PD-L1-CAR-T cells against cancer cells. 100% killing was observed in BxPC3 cells and >80% was observed in SKOV-3 cells with the combination.

The results showed that PD-L1 CAR-T cells can be used together with other CAR-T cells. The combination of PD-L1 CAR-T cells and CD24 CAR-T cells can be applied for co-inhibition of two pathways in vivo, when PD-L1 pathway is activated in tumor microenvironment.

### REFERENCES

1. Maus, M. V., Haas, A. R., Beatty, G. L., Albelda, S. M., Levine, B. L., Liu, X., Zhao, Y., Kalos, M., and June, C. H. (2013). T cells expressing chimeric antigen receptors can cause anaphylaxis in humans. Cancer Immunol Res 1, 26-31.
2. Maus, M. V., Grupp, S. A., Porter, D. L., and June, C. H. (2014). Antibody-modified T cells: CARs take the front seat for hematologic malignancies. Blood 123, 2625-2635.
3. Sgambato, A., Casaluce, F., Sacco, P. C., Palazzolo, G., Maione, P., Rossi, A., Ciardiello, F., and Gridelli, C. (2016). Anti PD-1 and PD-L1 Immunotherapy in the Treatment of Advanced Non-Small Cell Lung Cancer (NSCLC): A Review on Toxicity Profile and its Management. Curr Drug Saf 11, 62-68.
4. Boeye, A. (1986). Clonal isolation of hybridomas by manual single-cell isolation.

Methods Enzymol 121, 332-340.
5. Goluboskaya V, Wu L. Different Subsets of T Cells, Memory, Effector Functions, and CAR-T Immunotherapy Cancers (Basel). 2016 Mar. 15; 8(3). pii: E36. doi: 10.3390/cancers8030036. Review.

1. A monoclonal anti-human PD-L1 antibody or its antigen-binding fragment comprising V_{H} having the amino acid of SEQ ID NO: 3 and V_{L} having the amino acid of SEQ ID NO: 5, wherein the antibody binds to human PD-L1 protein.

2. A single-chain variable fragment (scFv) comprising V_{H} having the amino acid of SEQ ID NO: 3 and V_{L} having the amino acid of SEQ ID NO: 5, wherein the scFv binds to human PD-L1 protein.

3. The scFv of claim 2, further comprises a linker in between V_{H} and V_{L}.

4. The scFv of claim 2, which has the amino acid sequence of SEQ ID NO: 9.

5. A chimeric antigen receptor fusion protein (CAR) comprising from N-terminus to C-terminus:
(i) the scFv of claim 2,
(ii) a transmembrane domain,
(iii) at least one co-stimulatory domains, and
(iv) an activating domain.

6. The CAR of claim 5, wherein the scFv further comprises a linker in between V_{H} and V_{L}.

7. The CAR according to claim 5, wherein the co-stimulatory domain is CD28 or 4-1BB.

8. The CAR according to claim 5, wherein the activation domain is CD3 zeta.

9. The CAR of claim 5, which has the amino acid sequence of SEQ ID NO: 20 or 24.

10. A nucleic acid encoding the CAR of claim 5.

11. T cells or natural killer cells modified to express the CAR of claim 5.
